(19) United States
(12) Patent Application Publication
(10) Pub. No.: US 2026/0013703 A1
(43) Pub. Date: Jan. 15, 2026

(54) MEDICAL SYSTEMS, DEVICES, AND RELATED METHODS FOR DELIVERING FLUID INTO TISSUE

(71) Applicant: Boston Scientific Scimed, Inc., Maple Grove, MN (US)

(72) Inventors: Lauren Sfakis LYDECKER, Upton, MA (US); Carolina VILLARREAL, Hopedale, MA (US)

(73) Assignee: Boston Scientific Scimed, Inc., Maple Grove, MN (US)

(21) Appl. No.: 19/261,009

(22) Filed: Jul. 7, 2025

Related U.S. Application Data
(60) Provisional application No. 63/670,257, filed on Jul. 12, 2024.

(51) Int. Cl. A61B 1/00

(52) U.S. Cl. CPC A61B 1/00097; A61B 1/00042; A61B 1/00043; A61B 1/00066; A61B 1/00119; A61B 1/00124; A61B 1/00128; A61B 2560/0468; A61B 2562/0247

(57) ABSTRACT

A medical device includes a handle, a shaft, an injection device coupled to or a part of a distal end of the shaft, and a valve at the distal portion of the shaft. The handle includes a first fluid port and a second fluid port. The shaft includes a first lumen fluidly coupled to the first fluid port and a second lumen fluidly coupled to the second fluid port. The injection device includes an injection lumen. The valve is controllable to (1) fluidly couple the first lumen to the injection lumen, and block the second lumen from being fluidly coupled to the injection lumen; (2) fluidly couple the second lumen to the injection lumen, and block the first lumen from being fluidly coupled to the injection lumen; and (3) block both the first lumen and the second lumen from being fluidly coupled to the injection lumen.

### CROSS-REFERENCE TO RELATED APPLICATIONS

This application claims the benefit of priority to U.S. Provisional Application No. 63/670,257, filed on Jul. 12, 2024, which is incorporated by reference herein in its entirety.

### TECHNICAL FIELD

Various aspects of this disclosure generally relate to medical systems, devices, and related methods for delivering one or more fluids to tissue. Embodiments of the disclosure relate to medical systems, devices, and related methods of treating tissue by delivering or injecting one or more fluids into, under, and/or between layers of tissue with the distal element of the medical device.

### BACKGROUND

Medical devices, such as endoscopes or other suitable insertion devices, are employed for a variety of types of diagnostic and surgical procedures, such as endoscopy, laparoscopy, arthroscopy, gynoscopy, thoracoscopy, cystoscopy, etc. Many of these procedures involve delivering energy to tissue of an organ or a gland to treat lesions (e.g., tumors), infections, and the like. Examples of such procedures include Endoscopic Mucosal Resection (EMR), Endoscopic Sub-mucosal Resection (ESR), Endoscopic Sub-mucosal Dissection (ESD), polypectomy, mucosectomy, Peroral Endoscopic Myotomy (POEM), etc. In particular, such procedures may be carried out by inserting an insertion device into a subject's body through a surgical incision, or via a natural anatomical orifice (e.g., mouth, vagina, or rectum), and performing the procedure or operation at a treatment site with an auxiliary device inserted through the insertion device. Alternatively, the auxiliary device may be delivered to the treatment site without the insertion device.

In some procedures, a distal portion of a medical device is positioned between layers of tissue to cut, puncture, or perforate one or more layers of tissue or otherwise treat the tissue or the treatment site. In some aspects, a distal portion of the medical device is positioned between the mucosa and the muscularis layers in a portion of the gastrointestinal (GI) tract to reach a target or treatment site. Oftentimes, the positioning is achieved by injecting saline, a lifting agent, and/or one or more other fluids into the submucosa to separate the mucosa layer from the muscularis layer, for example, to help form a bleb. The medical device or an additional medical device may be used to cut, resect, or otherwise treat the treatment site (e.g., to remove the bleb). In some instances, it may be difficult to position the distal end of the medical device between the mucosa layer and the muscularis layer, and it may cause injury and/or lengthen the procedure if the distal end of the medical device is positioned in or between different layers of tissue. These concerns may increase the duration, costs, and risks of the medical procedure.

The systems, devices, and methods of this disclosure may rectify some of the deficiencies described above or address other aspects of the art.

### SUMMARY

Examples of this disclosure relate to, among other things, medical systems, devices, and methods for performing one or more medical procedures. For example, this disclosure relates to medical devices and methods for performing one of more procedures between layers of tissue. Additionally, in some examples, this disclosure relates to medical devices and methods for delivering fluid to a treatment site, for example, into or between tissue (e.g., layers of tissue). Furthermore, in some examples, this disclosure relates to medical systems, devices, and methods for lifting or otherwise manipulating tissue or one or more portions of the treatment site. Each of the examples disclosed herein may include one or more of the features described in connection with any of the other disclosed examples.

In one example, a medical device may include a handle, a shaft coupled to and extending from the handle, an injection device coupled to or a part of a distal end of the shaft, and a valve at the distal portion of the shaft. The handle may include a first fluid port and a second fluid port. The shaft may include a first lumen fluidly coupled to the first fluid port and a second lumen fluidly coupled to the second fluid port. The injection device may include an injection lumen. The valve may be controllable to (1) fluidly couple the first lumen to the injection lumen, and block the second lumen from being fluidly coupled to the injection lumen; (2) fluidly couple the second lumen to the injection lumen, and block the first lumen from being fluidly coupled to the injection lumen; and (3) block both the first lumen and the second lumen from being fluidly coupled to the injection lumen.

The medical device may include one or more of the following features. The medical device may further include an actuator to control a configuration of the valve. The actuator may be on the handle. The actuator may be a rotatable dial on the handle. The medical device may further include a control unit, and the control unit may be coupled to the handle via an umbilicus. The control unit may include a user interface or display configured to indicate whether it is desirable to inject one or more fluids through the injection device.

The medical device may further include a sensor. The sensor may be configured to detect and transmit one or more signals indicative of physical properties of tissue. The sensor may be positioned on or coupled to a portion of the injection device. The sensor may be configured to measure one or more of a density, a thickness, or a stiffness of a tissue. The sensor may be positioned on or coupled to a portion of one or more of the first lumen or the second lumen. The sensor may be a pressure sensor configured to detect a pressure of a fluid delivered through one or more of the first lumen or the second lumen.

The first lumen and the second lumen may be concentric such that the second lumen radially surrounds the first lumen. The second lumen may include a larger cross-sectional area than the first lumen. The injection device may be an electrode. The electrode may include a widened distal portion, and the injection lumen may be an electrode lumen. The handle may include an electrical connection to receive energy from an energy source. The handle may include a trigger. The trigger may be movable to control a position of the electrode relative to a distal end of the shaft. The injection device may be an injection needle, and the injection lumen may be a needle lumen.

In another aspect, a medical device may include a handle, a shaft coupled to an extending from the handle, an injection device coupled to or a part of the distal end of the shaft, and a valve at a distal portion of the shaft. The handle may include a first fluid port, a second fluid port, and at least one actuator. The shaft may include a first lumen fluidly coupled to the first fluid port and a second lumen fluidly coupled to the second port. The injection device may include an injection lumen. The valve may be controllable via the at least one actuator to (1) fluidly couple the first lumen to the injection lumen, and block the second lumen from being fluidly coupled to the injection lumen; (2) fluidly couple the second lumen to the injection lumen, and block the first lumen from being fluidly coupled to the injection lumen; and (3) block both the first lumen and the second lumen from being fluidly coupled to the injection lumen.

The medical device may further include a pressure sensor. The pressure sensor may be positioned on or coupled to a portion of one or more of the first lumen or the second lumen. The pressure sensor may be a pressure sensor configured to detect a pressure of a fluid delivered through one or more of the first lumen or the second lumen. The first lumen and the second lumen may be concentric such that the second lumen radially surrounds the first lumen. The second lumen may include a larger cross-sectional area than the first lumen.

In yet another aspect, a method of treating a treatment site may include positioning a distal end of a medical device to a treatment site. The medical device may include a shaft and an injection device at a distal end of the shaft. The shaft may include a first lumen and a second lumen. The medical device may include a valve at the distal end of the shaft that is movable to selectively couple the first lumen and the second lumen to the injection device. The method may also include positioning at least a portion of the injection device into one or more layers of tissue at the treatment site. The method may further include injecting one or more first fluids into the one or more layers of tissue at the treatment site through the first lumen, the valve, and the injection device. The method may also include injecting one or more second fluids into the one or more layers of tissue at the treatment site through the second lumen, the valve, and the injection device.

The method may further include, after injecting the one or more first fluids and before injecting the one or more second fluids, detecting one or more parameters of the tissue at the treatment site with one or more sensors. The method may further include repositioning the distal end of the medical device relative to the one or more layers at the treatment site.

It may be understood that both the foregoing general description and the following detailed description are exemplary and explanatory only and are not restrictive of the disclosure, as claimed. Any of the features described herein may be combined in any suitable combination or used separately.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 illustrates a perspective view of an exemplary medical device system, according to aspects of this disclosure.

FIG. 2 illustrates a longitudinal cross-sectional view of a distal portion of a medical device of the medical device system of FIG. 1, according to aspects of this disclosure.

FIG. 3 illustrates a longitudinal cross-sectional view of a distal portion of another medical device that may be included in the medical device system of FIG. 1, according to aspects of this disclosure.

FIGS. 4A-4C are schematic illustrations of a distal portion of a medical device and a treatment site, according to aspects of this disclosure.

FIGS. 5A and 5B are schematic illustrations of a distal portion of a medical device and tissue layers at a treatment site, according to aspects of this disclosure.

FIG. 6 is a flow diagram of an exemplary method, according to aspects of this disclosure.

### DETAILED DESCRIPTION

Examples of the disclosure include systems, devices, and methods for one or more of: facilitating and improving the efficacy, efficiency, and safety of treating and/or manipulating tissue when, for example, delivering one or more fluids into, under, and/or between layers of tissue during a medical procedure through the distal end of the medical device (e.g., an injection needle, an electrode, etc.); lifting, moving, or otherwise manipulating tissue; and/or otherwise treating tissue. Aspects of the disclosure may provide the user with the ability to deliver one or more fluids into, under, and/or between tissue layer(s) with a medical device. For example, aspects of this disclosure may help the user selectively deliver two or more fluids into, under, and/or between tissue layer(s) with the same medical device. Aspects of this disclosure may help the user accurately and/or appropriately position the distal end of the medical device relative to layers of tissue, for example, helping the user ensure that the fluid(s) are delivered to or between the correct layer(s) of tissue. Aspects of the disclosure may help the user penetrate a layer of tissue (e.g., a submucosal layer) to cause perforation or otherwise cut, cauterize, or otherwise treat tissue.

Aspects of the disclosure may help the user to lift, move, or otherwise manipulate tissue at or adjacent to a treatment site, which may help the user cut, resect, or otherwise remove additional tissue or other material. Aspects of the disclosure may help the user to lift, move, or otherwise manipulate tissue without having to switch or swap out fluid delivery devices. Additionally, aspects of the disclosure may help the user perform one or more of these treatments without requiring a larger insertion device and/or swapping out the fluid delivery devices, as a larger insertion device and/or swapping out fluid delivery devices may increase the procedure duration, may require multiple users, and/or may otherwise negatively affect the procedure. Moreover, aspects of the disclosure may help the user to lift, move, or otherwise manipulate tissue that has already been cut or otherwise treated, for example, such that that tissue may not interfere with or otherwise inhibit the cutting or treatment of additional tissue or other portions of the treatment site.

Embodiments of the disclosure may relate to systems, devices, and methods for performing various medical procedures and/or treating portions of the large intestine (colon), small intestine, cecum, esophagus, stomach, any other portion of the gastrointestinal tract, lungs, and/or any other suitable patient anatomy. Various embodiments described herein include single-use or disposable medical devices. Some aspects of the disclosure may be used in performing an endoscopic, bronchoscopic, ureteroscopic, colonoscopic, laparoscopic, arthroscopic, cystoscopic, gynoscopic, thoracoscopic, or other type of procedure. For example, the disclosed aspects may be used with endoscopes, duodenoscopes, bronchoscopes, ureteroscopes, colonoscopes, gastroscopes, endoscopic ultrasonography (“EUS”) scopes, laparoscopes, arthroscopes, cystoscopes, thoracoscopes, aspiration scopes, sheaths, catheters, diagnostic or therapeutic tools or devices, or other types of medical devices. One or more of the elements discussed herein could be metallic, plastic, or include a shape memory metal (such as, e.g., Nitinol), a shape memory polymer, a polymer, or any combination of biocompatible materials.

The terms “proximal” and “distal” are used herein to refer to the relative positions of the components of an exemplary medical device. When used herein, “proximal” refers to a position relatively closer to the exterior of the body of a subject or closer to a user, such as a medical professional, holding or otherwise using the medical device. In contrast, “distal” refers to a position relatively further away from the medical professional or other user holding or otherwise using the medical device, or closer to the interior of the subject's body. Proximal and distal directions are labeled with arrows marked “P” and “D”, respectively, throughout various figures. As used herein, the terms “comprises,” “comprising,” “has,” “having,” “includes,” “including,” or other variations thereof, are intended to cover a non-exclusive inclusion, such that a device or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value.

Reference will now be made in detail to examples of the disclosure described above and illustrated in the accompanying drawings. Wherever possible, the same reference numbers will be used throughout the drawings to refer to the same or like parts. It is noted that one or more aspects of the medical systems, devices, or methods discussed herein may be combined and/or used with one or more other aspects of the medical devices or methods discussed herein.

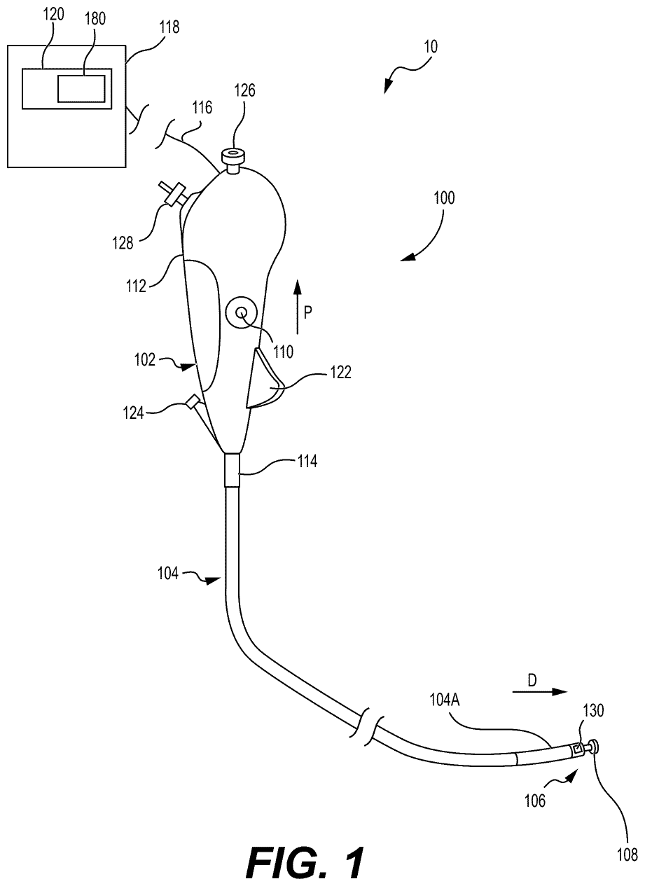

FIG. 1 illustrates a perspective view of an exemplary medical device system 10, including an exemplary medical device 100. Medical device 100 includes a handle 102 and a shaft 104. Shaft 104 includes a distal end 106. Medical device 100 also includes a tool or distal element, for example, an electrode 108 at distal end 106. In other aspects, the tool at distal end 106 may be a needle or other injection device. In any of these aspects, the tool at distal end 106 includes one or more lumens to deliver one or more fluids, for example, delivered through respective lumens in shaft 104. As discussed in detail below, distal end 106 may include a valve 130, regulator, or controller that controls which of the lumens in shaft 104 are open and/or closed, for example, to selectively deliver fluid(s) from the lumens in shaft 104.

In some aspects, medical device system 10 may include a control unit 118, for example, coupled to handle 102 via an umbilicus 116. Additionally, control unit 118 may include a user interface 120, for example, to control the configuration of valve 130. Alternatively or additionally, handle 102 or a proximal portion of shaft 104 may include an actuator 110, for example, manipulatable to control the configuration of valve 130. Actuator 110 may include one or more dials (e.g., rotatable dials), knobs, buttons, actuators, sliders, triggers, or other control or actuation mechanisms configured to toggle through or otherwise control the configuration of valve 130.

As shown in FIG. 1, handle 102 includes a handle body 112. A distal portion of handle body 112 may be coupled to a proximal portion of shaft 104 via a connector or strain relief element 114. Additionally, handle 102 includes one or more actuators or control mechanisms. For example, handle 102 may include an actuation mechanism, button, actuator, slider, or trigger 122, for example, movable relative to a distal portion of handle body 112. Trigger 122 may be movable, for example, slidable (e.g., longitudinally movable), pivotable, actuatable, or depressible, to control a position (e.g., extension and/or retraction) and/or energization of electrode 108. For example, sliding (e.g., distally), actuating, or depressing trigger 122 may extend electrode 108 from distal end 106 of shaft 104. In these aspects, electrode 108 may be extendable from distal end 106 of shaft 104 by approximately 5.0 mm to approximately 2.0 cm. Trigger 122 may be biased (e.g., spring-biased), such that trigger 122 may return to the proximal or non-depressed position once pressure on trigger 122 is no longer applied. Returning to the proximal or non-depressed position may also retract electrode 108 to the non-extended position.

Alternatively or additionally, in some aspects, sliding, actuating, or depressing trigger 122 may control the energization of electrode 108. For example, depressing trigger 122 may energize electrode 108, and releasing pressure on trigger 122 may terminate the energizing of electrode 108. In other aspects, the energization of electrode 108 may be controlled by a different button, actuator, or trigger either on or separate from handle body 112, for example, via a foot pedal.

Handle 102 also includes one or more connections and/or ports, for example, extending from or otherwise coupled to handle body 112. For example, handle 102 may include one or more of a first fluid port 124, a second fluid port 126, and/or an electrical connection 128. As discussed below, first fluid port 124 and/or second fluid port 126 may be fluidly connected to electrode 108, for example, via one or more respective lumens in handle 102 and shaft 104. Electrical connection 128 may be electrically connected to electrode 108, for example, via one or more conductive elements (e.g., wires, cables, filaments, rods, etc.) in handle 102 and shaft 104.

First fluid port 124, second fluid port 126, and/or electrical connection 128 may be positioned on respective portions of handle body 112. In some aspects, first fluid port 124 may be positioned on a distal portion of handle body 112, for example, adjacent or distal to trigger 122. Second fluid port 126 and/or electrical connection 128 may be positioned on a proximal portion of handle body 112, for example, proximal to trigger 122. Such positioning may facilitate an operator in introducing an appropriate fluid into ports 124 and/or 126. One or more of first fluid port 124 and/or second fluid port 126 may receive various fluids (e.g., water, saline(s), ORISE™ gel, fiducial marker(s) or fluid(s), another lifting agent, etc.) to be delivered via one or more respective lumens in shaft 104 and electrode 108, for example, to be delivered between two layers of tissue or otherwise to the treatment site. For example, one of first fluid port 124 and second fluid port 126 may receive a first fluid (e.g., water, saline, etc.), and the other of first fluid port 124 and second fluid port 126 may receive a second fluid (e.g., ORISE™ gel, a same or different saline, one or more fiducial markers or fluids, another lifting agent, etc.).

In any of these aspects, the lumens in shaft 104 and/or electrode 108 may converge. For example, shaft 104 may include at least two lumens, respectively fluidly connected to first fluid port 124 and second fluid port 126, and the at least two lumens may converge (e.g., in distal portion 104A of shaft 104) before being fluid connected to a single lumen in electrode 108. Additionally, the fluid connections between the lumens in shaft 104 and the lumen(s) in electrode 108 may be controlled by valve 130.

The delivery of the fluid to each of first fluid port 124 and/or second fluid port 126 may be controlled by a fluid delivery device (not shown), for example, a syringe, pump, etc. Moreover, electrical connection 128 may receive energy (e.g., cautery energy) from an energy source (not shown) to be delivered to electrode 108, for example, to cut, cauterize, penetrate, perforate, or otherwise treat tissue. In some aspects, as discussed above, the delivery of energy to electrical connection 128 may be controllable, for example, via a separate button or trigger, one or more foot pedals, etc. In other aspects, one or more of first fluid port 124, second fluid port 126, and/or electrical connection 128 may be connected to and receive fluid(s) and/or energy from control unit 118, for example, via umbilicus 116.

Furthermore, although not shown, handle 102 may include a strap or other connection element, for example, coupled to or otherwise extending from handle body 112. In some aspects, the strap or other connection element may help to allow for handle body 112 to be coupled to another medical device, for example, to a handle or other portion of a scope or other insertion device.

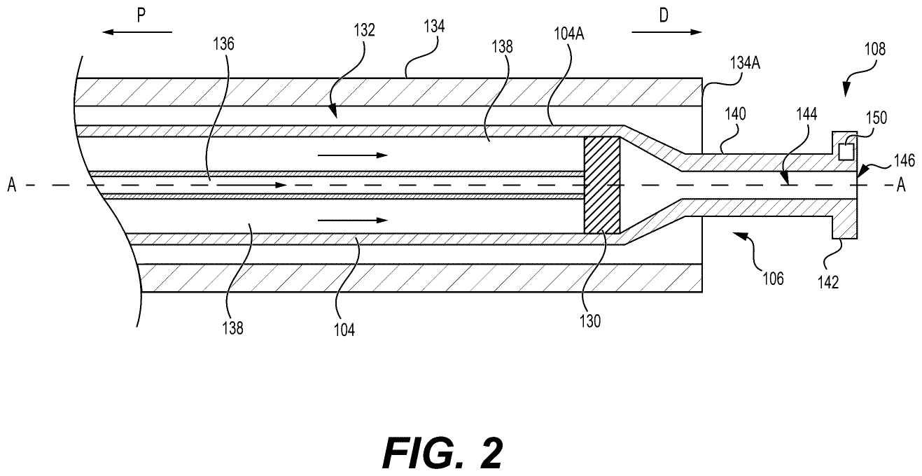

FIG. 2 illustrates a longitudinal cross-sectional view of distal portion 104A of shaft 104 and electrode 108. Although not shown, as mentioned above, electrode 108 may instead be a needle or other delivery device to deliver one or more fluids from shaft 104, for example, into tissue and/or between layers of tissue. Additionally, FIG. 2 illustrates distal portion 104A of shaft 104 inserted within a lumen 132 of an outer tube or sheath 134 or other insertion device. In some aspects, shaft 104 and/or electrode 108 may be moveable (e.g., longitudinally movable or slidable) relative to sheath 134, for example, within lumen 132 of sheath 134. For example, one or more portions of handle 102 (e.g., handle body 112 and/or trigger 122) may help to control a position of shaft 104 and/or electrode 108 relative to a distal end 134A of sheath 134.

As shown, shaft 104 includes at least two lumens, for example, a first lumen 136 and a second lumen 138. First lumen 136 may deliver a first fluid, for example, saline. The first fluid may be delivered distally through first lumen 136, as shown by the arrow within first lumen 136. For example, a proximal end (not shown) of first lumen 136 may be coupled to a first fluid source, for example, via first fluid port 124 (FIG. 1). Second lumen 138 may deliver a second fluid, for example, a lifting agent (e.g., ORISE™ Gel, a polysaccharide-based hydrogel, saline, one or more fiducial markers or fluids, etc.). The second fluid may be delivered distally through second lumen 138, as shown by the arrows within second lumen 138. For example, a proximal end (not shown) of second lumen 138 may be coupled to a second fluid source, for example, via second fluid port 126. Additional lifting agents and fluid sources that may be applicable to disclosure are discussed in US Pre-Grant Publication No. 2023/0355834, which is incorporated herein by reference.

In some aspects, first lumen 136 and second lumen 138 may be concentric. For example, as shown in FIG. 2, first lumen 136 may be radially within second lumen 138 (e.g., relative to a longitudinal axis A of shaft 104). Stated another way, second lumen 138 may be an outer lumen that radially surrounds first lumen 136. Alternatively, although not shown, first lumen 136 and second lumen 138 may be adjacent lumens (e.g., may extend alongside one another but not be concentric). In any of these aspects, one of first lumen 136 or second lumen 138 may be different sizes (e.g., have different lateral cross-sectional areas) and/or shapes. For example, a lateral cross-sectional area of first lumen 136 may be smaller than a lateral cross-sectional area of second lumen 138. In another aspect, first lumen 136 may be slit-shaped or generally rectangular, while second lumen 138 may be generally circular, or vice versa. In some aspects, the first fluid delivered through first lumen 136 may be less viscous than the second fluid delivered through second lumen 138.

In these aspects, first lumen 136 and second lumen 138 may be different sizes, for example, to receive, support, or otherwise convey different viscous fluids. In some aspects, first lumen 136 may be used to deliver saline, and second lumen 138 may be used to deliver the lifting agent. In this example, second lumen 138 may be approximately two, three, etc. times larger in lateral cross-sectional area and/or diameter than first lumen 136. Furthermore, in some aspects, one or more of first lumen 136 or second lumen 138 may be tapered, for example, tapering in lateral cross-sectional area or diameter from at least a proximal end or portion to a distal end or portion (e.g., with a consistent area or diameter over a proximal portion, and with a taper in a distal portion). In some aspects, the one or more tapered portions of first lumen 136 or second lumen 138 may help to control or otherwise limit the rate or speed at which the fluid(s) is/are delivered to the treatment site.

Electrode 108 may be energized to puncture, perforate, cut, resect, cauterize, remove, or otherwise treat tissue or other portions of a treatment site. In some aspects, as shown in FIG. 2, electrode 108 may include an electrode shaft 140 and a widened distal portion 142. In other aspects, electrode 108 may be generally cylindrical, that is, without a widened distal portion 142. Alternatively, electrode 108 may be one or more other shapes. In any of these aspects, electrode 108 includes an electrode lumen 144 (FIG. 2) or a central lumen that terminates in one or more electrode outlets or electrode openings 146 to inject or otherwise deliver fluid to the treatment site, for example, into or between layers of tissue. Electrode opening(s) 146 may deliver fluid distally, for example, along or parallel to the longitudinal axis A. Alternatively, electrode opening(s) 146 may deliver fluid at an angle relative to (e.g., perpendicularly to) longitudinal axis A. Although not shown, one or more portions of electrode 108 may include an insulative material (e.g., a ceramic, glass, polymeric material, etc.). In some aspects, electrode 108 may be movable (e.g., longitudinally extendable and retractable) relative to shaft 104, for example, extendable and/or retractable longitudinally relative to distal end 106 of shaft 104. Alternatively or additionally, shaft 104 may be movable relative to sheath 134 to extend and/or retract electrode 108.

In some aspects, one or more portions of electrode 108 may include one or more sensors 150. For example, a distal portion (e.g., widened distal portion 142) of electrode 108 may include sensor 150. Alternatively or additionally, sensor(s) 150 may be positioned within or adjacent to electrode lumen 144. In yet another alternative, sensor(s) 150 may be positioned within one or more of first lumen 136 or second lumen 138. Furthermore, in another alternative or additional feature, sensor(s) 150 may be positioned within handle body 112, first fluid port 124, second fluid port 126, the other fluidic connections, and/or capital equipment (e.g., in umbilicus 116 and/or control unit 118).

As discussed in detail below, sensor(s) 150 may help to detect a pressure or other aspect of the fluid being delivered by medical device 100, for example, the pressure within first lumen 136, second lumen 138, electrode lumen 144, and/or one or more other positioned on medical device 100. For example, fluid delivered between a first pair of layers of tissue may help to separate the layers of tissue, such that the fluid is delivered at a lower pressure. However, if fluid is delivered between a second pair of layer of tissue (or into a single layer of tissue) may not separate the layers of tissue, such that the fluid is delivered at a higher pressure. Alternatively or additionally, sensor(s) 150 may help to detect a tissue density and/or thickness, for example, based on a stiffness or other physical property of the tissue interacting with electrode 108. For example, different portions of a colon include different physical or mechanical properties (e.g., different stiffnesses). In these aspects, different tissue layers (e.g., mucosa, submucosa, muscle propria, etc.) have different tissue stiffnesses and thicknesses. As such, sensor(s) 150 may be able to discern between mucosa and submuscosa layers, for example, to allow for lifting agent injection. However, if sensor(s) 150 is/are positioned into the muscularis or muscle propria (e.g., with increased tissue stiffness, higher physical resistance, etc. feedback to sensor(s) 150), then sensor(s) 150 may identify and/or signal that one or more portions of electrode 108 has gone too deep into the tissue. As such, sensor(s) 150 may signal or otherwise prevent electrode 108 from delivering materials or fluids, helping to prevent the materials or fluid from being injected into the wrong layer of tissue at the treatment site. Alternatively or additionally, sensor(s) 150 may be configured to detect an intraluminal pressure, for example, to help prevent materials or fluids from being delivered into an inappropriate portion or layer of tissue at the treatment site.

Sensor(s) 150 may be connected (e.g., via one or more wired or wireless connections) to one or more processors, for example, in handle 102 and/or control unit 118, and one or more of handle 102 and/or control unit 118 may provide one or more indications to the user concerning the tissue interacting with electrode 108. For example, control unit 118 may display one or more indications on user interface 120. In some aspects, control unit 118 may include a display 180, for example, as a part of user interface 120 or separate from user interface 120 (as shown). Alternatively or additionally, handle 102 and/or control unit 118 may generate and/or emit one or more other signals or feedback to the user concerning the tissue interacting with electrode 108. For example, the one or more signals or feedback may include one or more visual indications, one or more audible indications, tactile or haptic feedback, etc. In any of these aspects, sensor(s) 150 may help to detect a position of electrode 108 (or other fluid injection end effector) relative to one or more layers of tissue.

Valve 130 may selectively controllable to either (1) block or impede distal ends of both first lumen 136 and second lumen 138; (2) block or impede the distal end of first lumen 136, while allowing the distal end of second lumen 138 to be fluidly connected to electrode 108 (e.g., electrode lumen 144); or (3) block or impede the distal end of second lumen 138, while allowing the distal end of first lumen 136 to be fluidly connected to electrode 108 (e.g., electrode lumen 144). For example, valve 130 may be controllable via actuator 110 on handle 102. As mentioned, actuator 110 may include one or more dials, knobs, buttons, actuators, sliders, triggers, or other control or actuation mechanisms configured to toggle through or otherwise control the configuration of valve 130.

For example, with actuator 110 in a first configuration, valve 130 may block or impede distal ends of both first lumen 136 and second lumen 138. With actuator 110 in a second configuration different from the first configuration, valve 130 may block or impede the distal end of first lumen 136, while allowing the distal end of second lumen 138 to be fluidly connected to electrode 108 (e.g., electrode lumen 144). Additionally, with actuator 110 in a third configuration different from the first configuration and the second configuration, valve 130 may block or impede the distal end of second lumen 138, while allowing the distal end of first lumen 136 to be fluidly connected to electrode 108 (e.g., electrode lumen 144). Actuator 110 may be rotated, depressed, slid (e.g., longitudinally or laterally), or otherwise manipulated to transition between the respective configurations of actuator 110, and thus transition valve 130 between the various configurations of valve 130.

In some aspects, although not shown, medical device 100 may include a plurality of actuators to control valve 130 between the various configurations. For example, medical device 100 may include a first actuator on handle body 112 that is selectable (e.g., rotatable, depressible, slidable, or otherwise manipulatable) to transition valve 130 into the first configuration. Additionally, medical device 100 may include a second actuator on handle body 112 that is selectable (e.g., rotatable, depressible, slidable, or otherwise manipulatable) to transition valve 130 into the second configuration. Moreover, medical device 100 may include a third actuator on handle body 112 that is selectable (e.g., rotatable, depressible, slidable, or otherwise manipulatable) to transition valve 130 into the third configuration. Alternatively or additionally, medical system 10 may include one or more actuators (e.g., separate from handle 102) to control and/or transition valve 130 between the various configurations. For example, control unit 118 (e.g., user interface 120) may include one or more actuators to control and/or transition valve 130 between the various configurations. In some aspects, control unit 118 may be or include one or more buttons, sliders, or one or more other actuators positioned outside of or separate from handle 102, for example, as part of a foot pedal.

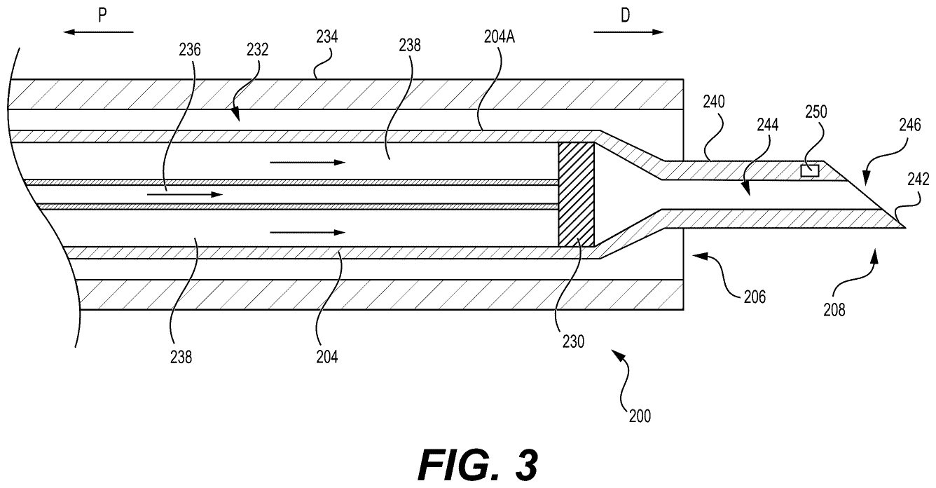

FIG. 3 illustrates a distal portion 206 of another medical device 200, for example, including a shaft 204 (e.g., with distal portion 204A) and an injection needle 208. Elements or portions of medical device 200 that are similar to medical device 100 are shown by 100 added to the respective element numbers, unless otherwise noted. For example, injection needle 208 may be coupled to shaft 204, as discussed above with respect to electrode 108 and shaft 104, for example, to a distal end 204A of shaft 204. Alternatively, distal end 204A of shaft 204 may form injection needle 208. Specifically, shaft 204 includes a first lumen 236 and a second lumen 238, and injection needle 208 may include an injection needle lumen 244. A first fluid may be delivered distally through first lumen 236, as shown by the arrow within first lumen 236. Similarly, a second fluid may be delivered distally through second lumen 238, as shown by the arrows within second lumen 238. First lumen 236 and second lumen 238 may be selectively fluidly connected to injection needle lumen 208A via a valve 230, as discussed above for valve 130 of medical device 100. For example, a handle (not shown) may include one or more actuators configured to control the configuration of valve 230.

As shown in FIG. 3, injection needle 208 may include a shaft portion 240 and may also include a tapered or pointed tip, for example, at a distal end 242 of injection needle 208. Injection needle 208 also includes one or more injection needle openings 246, for example, at a distal end of injection needle lumen 244. Additionally, as discussed above, shaft 204 and/or injection needle 208 may be movable (e.g., longitudinal movable or slidable) relative to an outer tube or sheath 234, for example, within a lumen 232 of sheath 234. Furthermore, one or more portions of injection needle 208 may include one or more sensors 250, as discussed above for sensors 150.

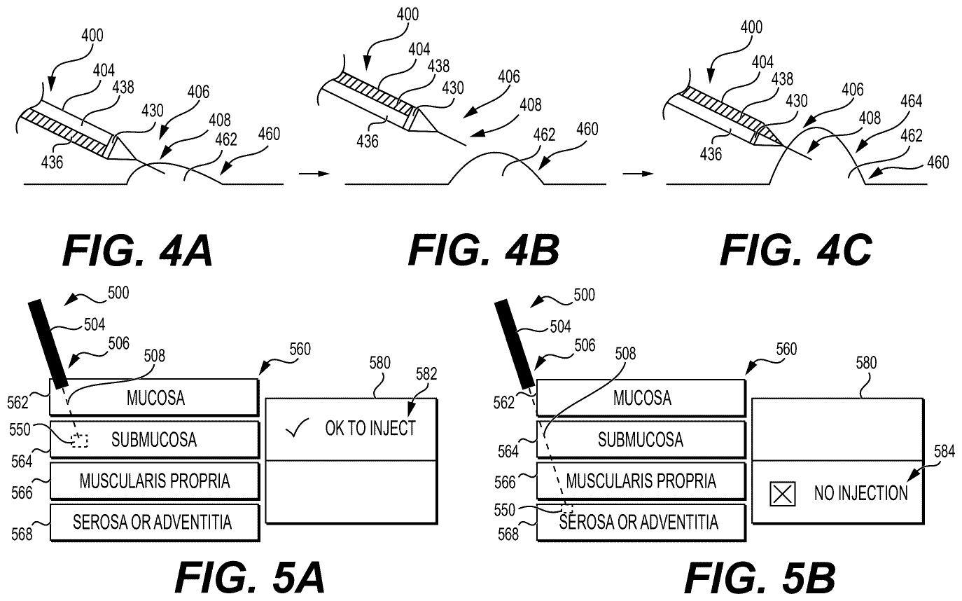

FIGS. 4A-4C schematically illustrate a distal portion 406 of a medical device 400 and a treatment site 460 in different configurations. Elements or portions of medical device 400 that are similar to medical device 100 are shown by 300 added to the respective element numbers, unless otherwise noted. Similarly, elements or portions of medical device 400 that are similar to medical device 200 are shown by 200 added to the respective element numbers, unless otherwise noted. Accordingly, FIGS. 4A-4C illustrate medical device 400 schematically, for example, a simplified version of a medical device (e.g., medical device 100 or medical device 200).

Medical device 400 includes a shaft portion 404 and an injection portion 408. Shaft portion 404 includes at least two lumens, for example, a first lumen 436 and a second lumen 438 (e.g., similar to first lumens 136, 236 and second lumens 138, 238). First lumen 436 and second lumen 438 of shaft portion 404 may be selectively fluidly connected to injection portion 408, for example, via a valve 430 (e.g., similar to valves 130, 230). Additionally, injection portion 408 may be an electrode (e.g., electrode 108), an injection needle (e.g., injection needle 208), or another injection element. Although not shown, shaft portion 404 may be coupled to a handle portion that includes ports fluidly connected to first lumen 436 and second lumen 438, respectively.

As shown in FIG. 4A, first lumen 436 may be fluidly connected to injection portion 408 via valve 430. In this aspect, as shown, a distal portion of injection portion 408 may be positioned within a portion of tissue 462 at treatment site 460. Accordingly, the first fluid (e.g., saline) may be delivered from first lumen 436, through injection portion 408, and into tissue 462. In this aspect, the first fluid may help to separate layers of tissue (e.g., to help separate a mucosa layer and a submucosa layer).

As shown in FIG. 4B, injection portion 408 may be removed from the tissue at treatment site 460. With injection portion 408 removed, valve 430 may be controlled to modify the fluidic connections of shaft portion 404. For example, instead of first lumen 436 being fluidly connected to injection portion 408, second lumen 438 may be fluidly connected to injection portion 408. In some aspects, the user may observe (e.g., perform a visual check) of treatment site 460 after the injection of the first fluid and/or before the injection of the second fluid. Alternatively or additionally, one or more sensors (e.g., one of sensors 150, 250 discussed above and/or sensor 550 discussed below) may help the user observe treatment site 460 and/or help in a reinsertion and/or repositioning of injection portion 408. It is noted, however, that in some aspects, valve 430 may be controlled to modify the fluidic connections of shaft portion 404 without removing injection portion 408 from the tissue at treatment site 460. Similarly, in some aspects, valve 430 may be controlled to modify the fluidic connections of shaft portion 404 without repositioning injection portion 408 from the tissue at treatment site 460. For example, once injection portion 408 is appropriately positioned, injection portion 408 may be maintained in the same position at treatment site 460. Additionally, valve 430 may be transitioned such that the second fluid may be injected to the appropriate position at treatment site 460.

As shown in FIG. 4C, second lumen 438 may be fluidly connected to injection portion 408 via valve 430. In this aspect, as shown, a distal portion of injection portion 408 may be positioned within the portion of tissue 462 at treatment site 460. Accordingly, the second fluid (e.g., lifting agent) may be delivered from second lumen 438, through injection portion 408, and into tissue 462. In this aspect, the second fluid may help to separate the layers of tissue. In some aspects, the second fluid may help to further separate the layers of tissue, for example, to help create and/or maintain a bleb 464.

It is noted that one or more steps of FIGS. 4A-4C may be repeated as many times as necessary, for example, to help form and/or remove the tissue 462 that forms bleb 464 and/or otherwise treat treatment site 460. For example, one or more of shaft portion 404, injection portion 408, an outer sheath (not shown) may be advanced distally or otherwise manipulated relative to the treatment site 460 to inject one or more fluids into, below, and/or between layers of tissue to help form and/or maintain one or more blebs 464. Additionally or alternatively, injection portion 408 may be energized (e.g., as discussed above with respect to electrode 108) to help apply energy, cut, cauterize, or otherwise treat tissue 462 at treatment site 460.

FIGS. 5A and 5B schematically illustrate a distal portion 506 of another medical device 500 and a treatment site 560. In some aspects, as mentioned above, medical device 500 may be coupled to (e.g., wired or wirelessly connected to) a user interface or display 580. As shown, treatment site 560 may include a plurality of layers of tissue. For example, if treatment site 560 is a portion of the gastrointestinal (GI) tract, treatment site 560 may include a mucosa layer 562 (e.g., epithelium, lamina propria, and muscular mucosae), a submucosa layer 564, a muscularis propria layer 566 (e.g., inner circular muscle layer, intermuscular space, and outer longitudinal muscle layer), a serosa or adventitia layer 568, etc. In some aspects, when treating various aspects of treatment site 560 (e.g., a polyp), it may be appropriate or desirable to deliver one or more fluids within or between some layers of tissue, but may be inappropriate or undesirable to deliver the one or more fluids within or between other layers of tissue.

As mentioned above, medical device 500 may include one or more sensors 550. For example, sensor(s) 550 may be positioned on one or more portions of injection portion 508. Alternatively or additionally, sensor(s) 550 may be positioned on one or more portions of an injection needle (e.g., injection needle 208). Furthermore, either alternatively or additionally, sensor(s) 550 may be positioned within one or more various portions of the first and second lumens, the handle, control unit, etc., as discussed above. Moreover, sensor(s) 550 be similar to sensors 150, 250, as discussed above, and may help to detect a pressure of the fluid being delivered via medical device 500, the density, thickness, and/or stiffness of the tissue that the distal end of medical device 500 is interacting with, etc. In these aspects, sensor(s) 550 may help identify the layer(s) of tissue that the distal end of medical device 500 is interacting with.

For example, with the distal end (e.g., injection portion 508) of medical device 500 inserted into tissue at treatment site 560, saline may first be injected (e.g., via a first lumen of shaft portion 504). Sensor(s) 550 may measure the fluid pressure at which the saline is delivered, and the one or more processors may receive signals from sensor(s) 550 and help to determine where the distal end of medical device 500 is positioned in relation to the layers of tissue at treatment site 560. Once the distal end of medical device 500 is appropriately positioned (e.g., as confirmed by sensor(s) 550), the user may deliver a lifting agent (e.g., via a second lumen of shaft portion 504) into, under, or between layer(s) of tissue.

In another example, with the distal end (e.g., injection portion 508) of medical device 500 inserted into tissue at treatment site 560, the one or more sensors 550 may help to measure a density, thickness, and/or stiffness of an adjacent tissue layer. As mentioned, sensor(s) 550 may be connected to one or more processors. The one or more processors may receive signals from sensor(s) 550 and help to determine where the distal end of medical device 500 is positioned in relation to the layers of tissue at treatment site 560. Once the distal end of medical device 500 is appropriately positioned (e.g., as confirmed by sensor(s) 5150), the user may deliver a lifting agent into, under, or between layer(s) of tissue.

In any of these aspects, as discussed above, sensor(s) 550 may be coupled to user interface or display 580. Although not shown, one or more control units, processors, etc. may help process one or more signals from sensor(s) and/or otherwise communicate, transmit, or send signals from sensor(s) 550 to user interface (e.g., user interface 120) or display 580. In some aspects, it may be appropriate or desirable to inject one or more fluids into, between, and/or adjacent to mucosa layer 562 and/or submucosa layer 564. Furthermore, it may be inappropriate or undesirable to inject one or more fluids into, between, and/or adjacent to muscularis propria layer 566 and/or serosa or adventitia layer 568.

In any of these aspects, user interface or display 580 may display one or more indications that correspond to whether it is appropriate or desirable to inject one or more fluids into or between layers of tissue based on signals received from sensor(s) 550. For example, if it is appropriate or desirable to inject the one or more fluids, user interface or display 580 may display a first indication 582, for example, including one or more of a checkmark, may be green, and/or may display “OK to inject.” Additionally, if it is inappropriate or undesirable to inject one or more fluids, user interface or display 580 may display a second indication 584, for example, including one or more of an X, may be red, and/or may display “NO injection.” Moreover, as discussed above, user interface or display 580, the handle of medical device 500, and/or one or more other aspects of medical device 500 may generate and/or emit one or more other signals or feedback to the user concerning the tissue interacting with the distal end of medical device 500, for example, one or more visual indications, one or more audible indications, tactile or haptic feedback, etc. In these aspects, sensor(s) 550 may help the user identify the layers of tissue at treatment site 560 and/or whether it is appropriate or inappropriate to inject one or more fluids into, underneath, and/or between one or more layers of tissue at treatment site 560.

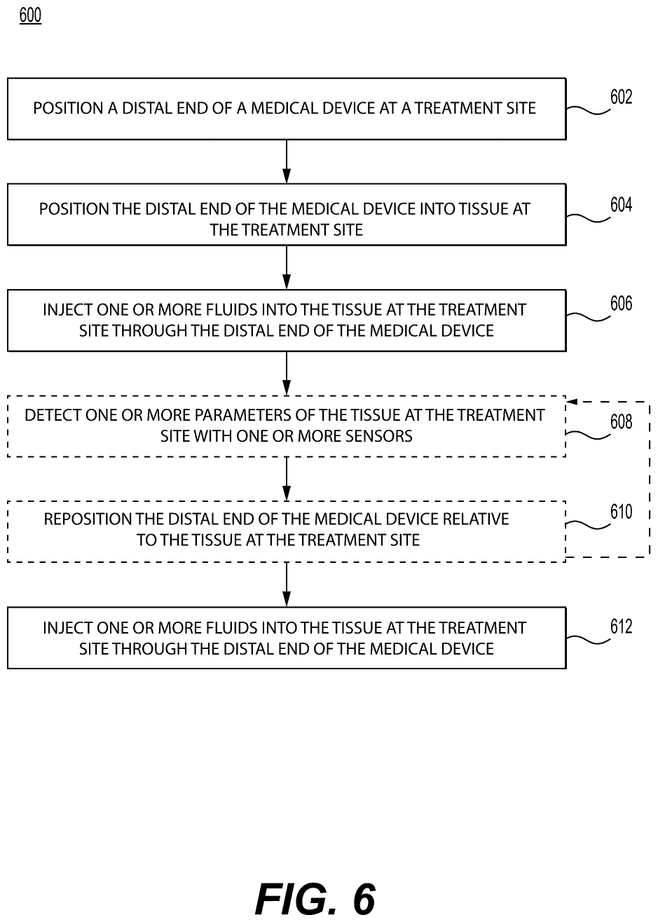

FIG. 6 illustrates a method 600 that may be performed with any of the medical systems 10 and medical devices 100, 200, 400, 500. A step 602 includes positioning a distal end of a medical device at a treatment site. For example, as discussed above, the distal end of the medical device may be delivered to the treatment site via an insertion device (e.g., sheath 134, 234). For example, the treatment site may be a portion of the GI tract, and the insertion device may be an endoscope.

Next, a step 604 includes positioning the distal end of the medical device into tissue at the treatment site. For example, as discussed above, the distal end of the medical device may be an electrode (e.g., electrode 108), a needle (e.g., injection needle 208), or other injection device. Moreover, the treatment site may be a portion of the GI tract, and the tissue may include mucosa layer 562, submucosa layer 564, muscularis propria layer 566, serosa or adventitia layer 568, etc. Step 604 may include positioning the distal end of the medical device into, under, or between one or more layers of tissue at the treatment site. Positioning the distal end of the medical device may include one or more manipulations of the insertion device (e.g., deflection and/or extension of sheath 134, 234), one or more manipulations of the medical device (e.g., actuation of trigger 122 on handle body 112), etc. Additionally or alternatively, positioning the distal end of the medical device may include energizing the distal end, for example, energizing electrode 108 via energy delivered to handle 102 via electrical connection 128.

Next, a step 606 includes injecting one or more fluids into the tissue at the treatment site through the distal end of the medical device. For example, as discussed above, injecting one or more fluids may include controlling one or more fluid sources (e.g., a syringe coupled to handle body 112). Injecting one or more fluids may include delivering a fluid (e.g., saline) into first fluid port 124, through first lumen 136, and out of the distal end of the medical device. For example, actuator 110 on handle body 112 may control valve 130, such that first lumen 136 is fluidly connected to the injection device (e.g., electrode lumen 144 of electrode 108, injection needle lumen 244 of injection needle 208, etc.). In some aspects, step 606 may include observing the treatment site during and/or after the injection of the one or more fluids. For example, the insertion device may include one or more lighting and/or imaging elements, and the user may observe the treatment site to see whether the treatment site responds to the injection (e.g., layers of tissue separate and/or form a bleb). In these aspects, separation of layers of tissue and/or the formation of a bleb (e.g., as shown in FIGS. 4A-4C) may be indicative of the distal end of the medical device being properly positioned (e.g., between mucosa layer 562 and submucosa layer 564).

In some aspects, method 600 may include an optional step 608, which may include detecting one or more parameters of the tissue at the treatment site with one or more sensors. For example, as discussed above, sensor(s) 150, 550 may be positioned on or one or more portions of medical device 100. Additionally, sensor(s) 150, 550 may be in communication with one or more processors, which may help to determine a position of the distal end of the medical device relative to the treatment site (e.g., which layer of tissue that the distal end is interacting with). In some aspects, optional step 608 may include one or more outputs, indications, feedback, etc. (e.g., visual, audible, haptic, tactile, etc.) as to whether the distal end of the medical device is appropriately positioned relative to the layers of tissue at the treatment site. Alternatively or additionally, optional step 608 may include one or more displays or indications, for example, as discussed above with respect to FIGS. 5A and 5B.

Furthermore, in some aspects, method 600 may include another optional step 610, which may include repositioning the distal end of the medical device relative to the tissue at the treatment site. For example, if based on step 608, the distal end of the medical device is inappropriately positioned, step 610 may include proximally retracting or distally extending the distal end of the medical device (e.g., via trigger 122 on handle 102). In other aspects, the distal end of the medical device may be repositioned via movement (e.g., deflection, proximal retraction, distal extension, etc.) of the insertion device (e.g., sheath 134, 234). Then, in some aspects, method 600 may include returning to step 608 to again detect one or more parameters of the tissue at the treatment site with the one or more sensors, for example, to confirm that the distal end of the medical device is properly positioned for fluid delivery. Steps 608 and 610 may be repeated as many times as needed to properly position the distal end of the medical device.

Additionally, a step 612 includes injecting one or more other fluids into the tissue at the treatment site through the distal end of the medical device. In step 612, the one or more other fluids may be different from the one or fluids injected in step 606. For example, as discussed above, injecting one or more other fluids may include controlling one or more fluid sources (e.g., a syringe coupled to handle body 112). Injecting one or more fluids may include delivering a fluid (e.g., a lifting agent) through second lumen 138 (e.g., via second fluid port 126) and out of the distal end of the medical device. For example, actuator 110 on handle body 112 may control valve 130, such that second lumen 138 is fluidly connected to the injection device (e.g., electrode lumen 144 of electrode 108, injection needle lumen 244 of injection needle 208, etc.). In some aspects, step 612 may include observing the treatment site during and/or after the injection of the one or more fluids. For example, the insertion device may include one or more lighting and/or imaging elements, and the user may observe the treatment site to see whether the treatment site responds to the injection (e.g., layers of tissue separate and/or form a bleb). In these aspects, separation of layers of tissue and/or the formation of a bleb (e.g., as shown in FIGS. 4A-4C) may be indicative of the distal end of the medical device being properly positioned (e.g., between mucosa layer 562 and submucosa layer 564). In some aspects, the one or more other fluids (e.g., a lifting agent) may help to maintain a bleb for a longer period of time than the one or more fluids (e.g., saline) injected in step 606. For example, the one or more other fluids may help to maintain a separation of layers of tissue for a longer period of time.

The steps of method 600 may be performed as many times as necessary to treat the treatment site. For example, the user may form a bleb at the treatment site a first time, and then may reform the bleb at a second time. Alternatively or additionally, the user may use the medical device to form a plurality of blebs in the tissue at the treatment site.

Various aspects discussed herein may allow for one or more medical devices (e.g., medical device 100) to be delivered to a treatment site, for example, alone or via a scope or insertion device, to form a bleb, perform endoscopic submucosal dissection (“ESD”), or otherwise treat a treatment site. Various aspects discussed herein may help to allow for the user to deliver one or more fluids into, under, or between one or more layers of tissue. Various aspects discussed herein may help the user deliver a first fluid (e.g., saline) into, under, or between one or more layers of tissue, and then deliver a second fluid (e.g., a lifting agent) into, under, or between one or more layers of tissue. The first and second fluids may be delivered without removing the medical device from the treatment site. Moreover, various aspects discussed herein may help determine the position of the distal end of the medical device (e.g., electrode 108, injection needle 208, etc.) relative to one or more layers of tissue at the treatment site. Various aspects discussed herein may help to allow for the user to more effectively or efficiently apply energy, cut, cauterize or otherwise treat tissue, for example, with a reduced likelihood of cut tissue or other aspects of the treatment site interfering with the treatment. Additionally, various aspects discussed herein may help to improve the efficacy of treatment and/or recovery from a procedure, for example, a procedure to treat a treatment side. Various aspects discussed herein may help to reduce and/or minimize the duration of the procedure.

While principles of this disclosure are described herein with reference to illustrative aspects for various applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

We claim:

1. A medical device, comprising:
a handle, wherein the handle includes:
a first fluid port; and
a second fluid port; and
a shaft coupled to and extending from the handle, wherein the shaft includes:
a first lumen, wherein the first lumen is fluidly coupled to the first fluid port; and
a second lumen, wherein the second lumen is fluidly coupled to the second fluid port;
an injection device coupled to or a part of a distal end the shaft, wherein the injection device includes an injection lumen; and
a valve at a distal portion of the shaft, wherein the valve is controllable to (1) fluidly couple the first lumen to the injection lumen, and block the second lumen from being fluidly coupled to the injection lumen; (2) fluidly couple the second lumen to the injection lumen, and block the first lumen from being fluidly coupled to the injection lumen; and (3) block both the first lumen and the second lumen from being fluidly coupled to the injection lumen.

2. The medical device of claim 1, further comprising an actuator to control a configuration of the valve.

3. The medical device of claim 2, wherein the actuator is on the handle.

4. The medical device of claim 3, wherein the actuator is a rotatable dial on the handle.

5. The medical device of claim 1, further comprising a control unit, wherein the control unit is coupled to the handle via an umbilicus.

6. The medical device of claim 5, wherein the control unit includes a user interface or display configured to indicate whether it is desirable to inject one or more fluids through the injection device.

7. The medical device of claim 6, further comprising a sensor, wherein the sensor is configured to detect and transmit one or more signals indicative of physical properties of tissue.

8. The medical device of claim 7, wherein the sensor is positioned on or coupled to a portion of the injection device.

9. The medical device of claim 8, wherein the sensor is configured to measure one or more of a density, a thickness, or a stiffness of a tissue.

10. The medical device of claim 7, wherein the sensor is positioned on or coupled to a portion of one or more of the first lumen or the second lumen.

11. The medical device of claim 10, wherein the sensor is a pressure sensor configured to detect a pressure of a fluid delivered through one or more of the first lumen or the second lumen.

12. The medical device of claim 1, wherein the first lumen and the second lumen are concentric such that the second lumen radially surrounds the first lumen, and wherein the second lumen includes a larger cross-sectional area than the first lumen.

13. The medical device of claim 1, wherein the injection device is an electrode, wherein the electrode includes a widened distal portion, and wherein the injection lumen is an electrode lumen.

14. The medical device of claim 13, wherein the handle includes an electrical connection to receive energy from an energy source,
wherein the handle includes a trigger, and wherein the trigger is movable to control a position of the electrode relative to a distal end of the shaft.

15. The medical device of claim 1, wherein the injection device is an injection needle, and wherein the injection lumen is a needle lumen.

16. A medical device, comprising:
a handle, wherein the handle includes:
a first fluid port;
a second fluid port; and
at least one actuator; and
a shaft coupled to and extending from the handle, wherein the shaft includes:
a first lumen, wherein the first lumen is fluidly coupled to the first fluid port; and
a second lumen, wherein the second lumen is fluidly coupled to the second fluid port;
an injection device coupled to or a part of a distal end the shaft, wherein the injection device includes an injection lumen; and
a valve at a distal portion of the shaft, wherein the valve is controllable via the at least one actuator to (1) fluidly couple the first lumen to the injection lumen, and block the second lumen from being fluidly coupled to the injection lumen; (2) fluidly couple the second lumen to the injection lumen, and block the first lumen from being fluidly coupled to the injection lumen; and (3) block both the first lumen and the second lumen from being fluidly coupled to the injection lumen.

17. The medical device of claim 16, further comprising a pressure sensor, wherein the pressure sensor is positioned on or coupled to a portion of one or more of the first lumen or the second lumen, wherein the pressure sensor is a pressure sensor configured to detect a pressure of a fluid delivered through one or more of the first lumen or the second lumen.

18. The medical device of claim 16, wherein the first lumen and the second lumen are concentric such that the second lumen radially surrounds the first lumen, and wherein the second lumen includes a larger cross-sectional area than the first lumen.

19. A method of treating a treatment site, comprising:
positioning a distal end of a medical device to a treatment site, wherein the medical device includes a shaft and an injection device at a distal end of the shaft, wherein the shaft includes a first lumen and a second lumen, and wherein the medical device includes a valve at the distal end of the shaft that is movable to selectively couple the first lumen and the second lumen to the injection device;
positioning at least a portion of the injection device into one or more layers of tissue at the treatment site;
injecting one or more first fluids into the one or more layers of tissue at the treatment site through the first lumen, the valve, and the injection device; and
injecting one or more second fluids into the one or more layers of tissue at the treatment site through the second lumen, the valve, and the injection device.

20. The method of claim 19, further comprising:
after injecting the one or more first fluids and before injecting the one or more second fluids, detecting one or more parameters of the tissue at the treatment site with one or more sensors; and
repositioning the distal end of the medical device relative to the one or more layers at the treatment site.
